(19) United States
(12) Patent Application Publication
(10) Pub. No.: US 2013/0211456 A1
(43) Pub. Date: Aug. 15, 2013

(54) CONNECTOR AND FASTENER SYSTEM

(71) Applicants: Charles Anthony DICKINSON, Bartlett, TN (US); Alan REZACH, Atoka, TN (US)

(72) Inventors: Charles Anthony DICKINSON, Bartlett, TN (US); Alan REZACH, Atoka, TN (US)

(73) Assignee: Warsaw Orthopedic, Inc., Warsaw, IN (US)

(21) Appl. No.: 13/371,095

(22) Filed: Feb. 10, 2012

(51) Int. Cl. A61B 17/70; A61B 17/88

(52) U.S. Cl. 606/264; 606/279

(57) ABSTRACT

An implant system comprises a fastener including a proximal portion including an inner surface that defines a first cavity that defines a first axis and a distal portion configured for penetrating tissue and defining a longitudinal axis disposed transverse to the first axis. A connector extends between a first end and a second end configured for disposal in the first cavity. A receiver is attachable to the first end and includes an implant cavity defining a second axis. The implant cavity is rotatable about a third axis disposed transverse to the second axis such that an implant disposed in the implant cavity and posterior to the first end is rotatable in a first plane relative to the first end in a configuration for selective fixation with the first end. Methods of use are disclosed.

### TECHNICAL FIELD

The present disclosure generally relates to medical devices for the treatment of musculoskeletal disorders, and more particularly to a vertebral implant system and method that employs a connector and provides stabilization of vertebrae, which may include the sacroiliac region.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column. In another example, disorders of the sacroiliac joint can cause low back and radiating buttock and leg pain in patients.

Non-surgical treatments, such as medication, injection, mobilization, rehabilitation and exercise can be effective, however, may fail to relieve the symptoms associated with these disorders. Surgical treatment of these spinal disorders includes fusion, fixation, discectomy, laminectomy and implantable prosthetics. During surgical treatment, one or more rods may be attached via fasteners to the exterior of two or more vertebral members. Fasteners may also be attached to iliac bone. The present disclosure describes an improvement over these prior art technologies.

### SUMMARY

Accordingly, a vertebral implant system and method is provided. In one particular embodiment, in accordance with the principles of the present disclosure, the implant system comprises a fastener including a proximal portion and a distal portion. The proximal portion includes an inner surface that defines a first cavity that defines a first axis. The distal portion is configured for penetrating tissue and defines a longitudinal axis disposed transverse to the first axis. A connector extends between a first end and a second end configured for disposal in the first cavity. A receiver is attachable to the first end of the connector and includes an implant cavity defining a second axis. The implant cavity is rotatable about a third axis disposed transverse to the second axis such that an implant disposed in the implant cavity and posterior to the first end of the connector is rotatable in a first plane relative to the first end of the connector in a configuration for selective fixation with the first end of the connector.

In one embodiment, the implant system comprises an iliac bone screw including a posterior head and an anterior threaded shaft configured to penetrate bone. The posterior head includes an inner surface that defines a first cavity that defines a first axis. The inner surface includes axial splines oriented along the first axis and circumferentially disposed about the inner surface. The distal portion defines a longitudinal axis disposed transverse to the first axis. A connector extends between a first end and a second end. The first end includes a first disk having a first face defining an implant recess and a second face including a first splined surface. The second end includes an outer surface comprising axial splines circumferentially disposed thereabout. The axial splines of the posterior head are engageable with the axial splines of the second end in a configuration for selective angular fixation of the iliac bone screw with the connector. The second end further includes a first locking part comprising a circumferential flange. A receiver includes a second disk having a second splined surface that mates with the first splined surface. The receiver further includes a second locking part including a first arm, a second arm and a wishbone part. The wishbone part is disposable to engage and force the arms into fixation with the circumferential flange to lock the receiver with the connector. The receiver further includes an implant cavity defining a second axis. The implant cavity is rotatable about a third axis disposed transverse to the second axis such that a vertebral rod disposed in the implant cavity and posterior to the first end of the connector is selectively rotatable in a coronal plane of a body relative to the first end of the connector in a configuration for selective angular fixation with the first end of the connector.

In one embodiment, a method for treating a disorder is provided. The method includes the steps of providing an implant system having a fastener, a connector and a receiver. The fastener includes a proximal portion and a distal portion, the proximal portion including an inner surface that defines a first cavity that defines a first axis, the distal portion defining a longitudinal axis disposed transverse to the first axis. The connector extends between a first end and a second end configured for disposal in the first cavity. The receiver is attachable to the first end of the connector and includes an implant cavity defining a second axis rotatable about a third axis disposed transverse to the second axis. The distal portion of the fastener is attached with iliac bone. An implant is disposed in the implant cavity such that the implant is posterior to the first end of the connector. The implant is selectively rotated in a first plane relative to the first end of the connector. The receiver is then locked with the connector in a selected angular orientation of the implant relative to the connector.

### BRIEF DESCRIPTION OF THE DRAWINGS

The present disclosure will become more readily apparent from the specific description accompanied by the following drawings, in which:

FIG. 1 is a perspective view of one particular embodiment of an implant system in accordance with the principles of the present disclosure;

FIG. 2 is a perspective view of a connector of the implant system shown in FIG. 1 with parts separated;

FIG. 3 is a plan view of one embodiment of an implant system in accordance with the principles of the present disclosure attached with vertebrae and an iliac bone of a patient;

FIG. 4 is a perspective view of one embodiment of the implant system shown in FIG. 1;

FIG. 5 is a perspective view of a connector of the implant system shown in FIG. 4 with parts separated;

FIG. 6 is a perspective view of one embodiment of the implant system shown in FIG. 1;

FIG. 7 is a perspective view of a connector of the implant system shown in FIG. 6 with parts separated;

FIG. 8 is a perspective view of one embodiment of a connector of the implant system shown in FIG. 1;

FIG. 9 is a perspective view of the connector shown in FIG. 8 with parts separated;

FIG. 10 is a perspective view of one embodiment of a connector of the implant system shown in FIG. 1;

FIG. 11 is a perspective view of the connector shown in FIG. 10 with parts separated;

FIG. 12 is a perspective view of one embodiment of a connector of the implant system shown in FIG. 1; and

FIG. 13 is a perspective view of the connector shown in FIG. 12 with parts separated.

Like reference numerals indicate similar parts throughout the figures.

### DETAILED DESCRIPTION

The exemplary embodiments of the vertebral implant system and methods disclosed are discussed in terms of medical devices for the treatment of musculoskeletal disorders and more particularly, in terms of a vertebral implant system and method for treating a disorder. In one embodiment, the vertebral implant system includes a vertebral implant, such as, for example, a spinal rod and an iliac connector disposed with an iliac screw. It is envisioned that the implant system and methods disclosed may provide stability to a portion of the anatomy of a patient, such as, for example, vertebrae, a sacroiliac (SI) joint, iliac bone and maintain structural integrity while reducing stress on the SI joint and/or portions of the anatomy adjacent the SI joint.

In one embodiment, the implant system includes an iliac connector having a connecting element disposed anterior to a vertebral implant. This configuration disposes a substantial portion of the connector material anterior to a spinal rod to minimize a profile of the implant system posterior to the spinal rod. In one embodiment, the implant system includes a connector that can lock at different angles in a coronal plane of a body of a patient. This configuration facilitates connection of a spinal rod to an Iliac screw and avoids rod bending due to misalignment of the component parts.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

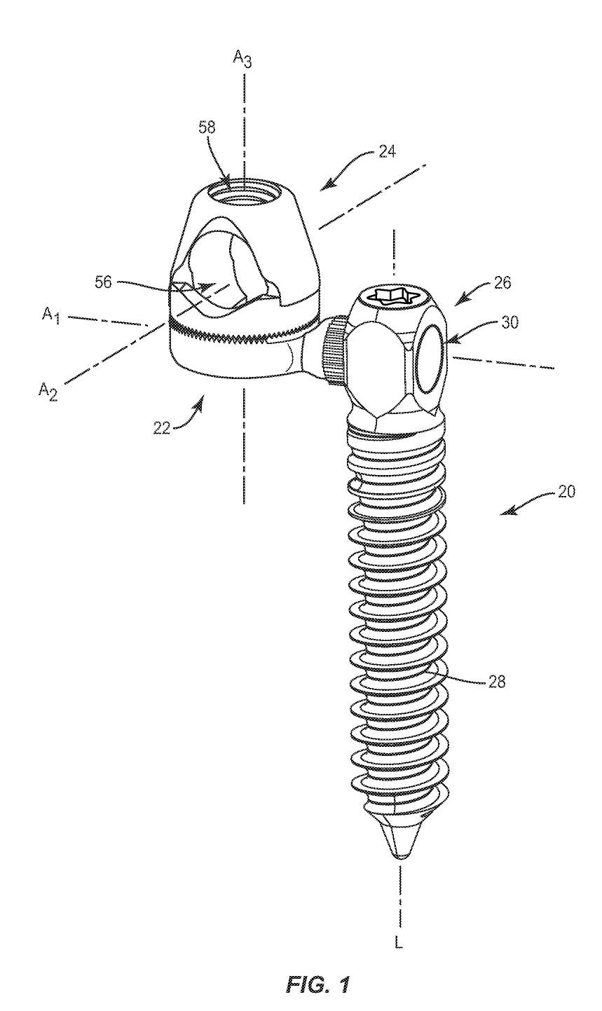

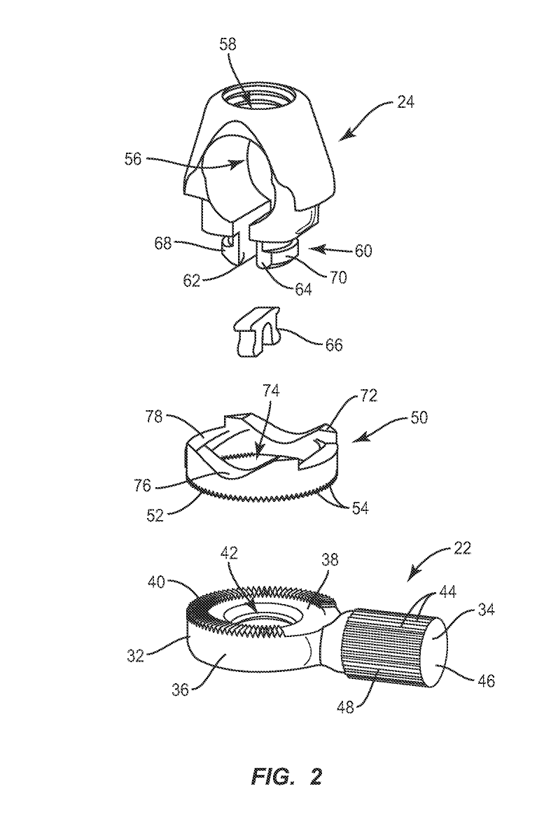

The following discussion includes a description of a surgical system including an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1 and 2, there is illustrated components of a surgical system including an implant system in accordance with the principles of the present disclosure.

The components of the implant system can be fabricated from biologically acceptable materials suitable for medical applications, including metals, synthetic polymers, ceramics and bone material and/or their composites, depending on the particular application and/or preference of a medical practitioner. For example, the components of the implant system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the implant system may have material composites, including the above materials, to achieve various desired characteristics such as strength, rigidity, elasticity, compliance, biomechanical performance, durability and radiolucency or imaging preference. The components of the implant system, individually or collectively, may also be fabricated from a heterogeneous material such as a combination of two or more of the above-described materials. The components of the implant system may be monolithically formed, integrally connected or include fastening elements and/or instruments, as described herein.

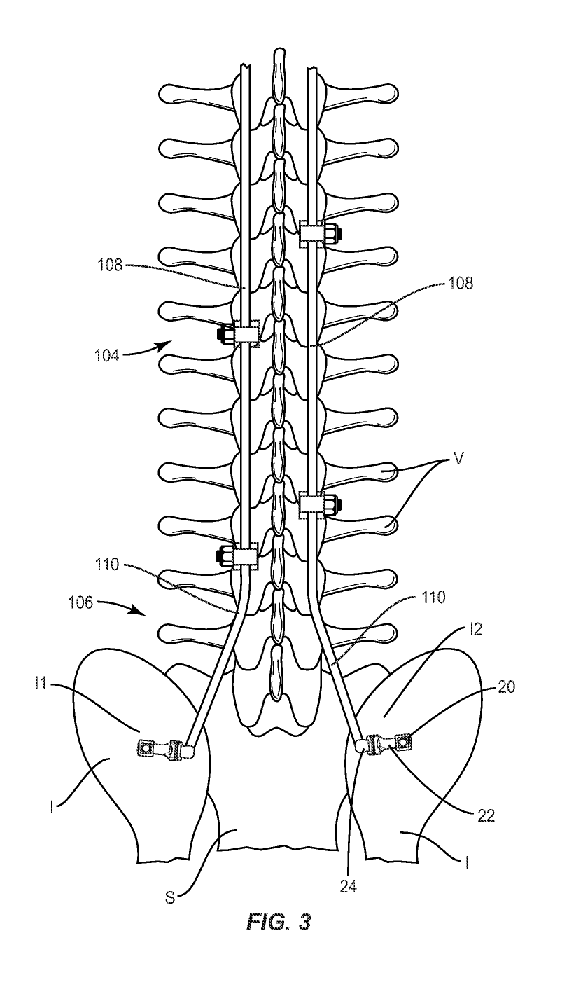

The implant system is configured for attachment to vertebrae and/or iliac bone (as shown, for example, in FIG. 3) during surgical treatment of a spinal disorder, examples of which being discussed herein. The implant system includes a fastener, such as, for example, an iliac bone screw 20, a connector 22 and a receiver 24. It is envisioned that the implant system may include one or a plurality of fasteners, connectors and/or receivers.

Bone screw 20 includes a proximal portion, such as, for example, a posterior head 26 and a distal portion, such as, for example, an anterior threaded shaft 28 configured to penetrate bone. Head 26 includes an inner surface that defines a first cavity 30 that defines a first axis A₁. First cavity 30 is configured to receive and movably support at least a portion of connector 22 such that connector 22 can translate axially within first cavity 30 along first axis A₁ through a first plane, such as, for example, a coronal plane of a body of a patient. It is contemplated that connector 22 may be disposed with bone screw 20 for relative movement thereto in orientations relative to first axis A₁, such as, for example, transverse, perpendicular and/or other angular orientations such as acute or obtuse, co-axial and/or may be offset or staggered. It is further contemplated that connector 22 may move relative to bone screw 20 in alternate planes relative to the body, such as, for example, transverse and/or sagittal planes of a body.

First cavity 30 has a tubular configuration that forms a passageway through bone screw 20. First cavity 30 is configured for receiving a portion of connector 22 that extends through head 26 of bone screw 20. It is envisioned that all or only a portion of first cavity 30 may be variously configured and dimensioned, such as, for example, oval, oblong, square, rectangular, polygonal, irregular, uniform, non-uniform, offset, staggered, tapered, consistent or variable, depending on the requirements of a particular application. In one embodiment, first cavity 30 may extend through only a portion of head 26 and not completely through.

First cavity 30 of head 26 includes axial splines 31 (not shown) circumferentially disposed about the inner surface of head 26 oriented along first axis A₁. First cavity 30 provides at least a portion of a mounting and alignment configuration for mating bone screw 20 with connector 22 and receiver 24 during a surgical treatment. Axial splines 31 in first cavity 30 include a plurality of individual spline members that extend in parallel relation about first cavity 30. Axial splines 31 in first cavity 30 mate with splines on connector 22 to align and mount bone screw 20 with connector 22 when bone screw 20 is connected with connector 22.

Anterior threaded shaft 28 defines a longitudinal axis L disposed transverse to first axis A₁. It is contemplated that anterior threaded shaft 28 may be disposed in other orientations relative to first axis A₁, such as, for example, perpendicular and/or other angular orientations such as acute or obtuse, co-axial and/or may be offset or staggered. Shaft 28 has a cylindrical cross section configuration and includes an outer surface having an external thread form. It is contemplated that the thread form may include a single thread turn or a plurality of discrete threads. It is further contemplated that other engaging structures may be located on shaft 28, such as, for example, a nail configuration, barbs, expanding elements, raised elements and/or spikes to facilitate engagement of shaft 28 with tissue, such as, for example, vertebrae and/or iliac bone.

It is envisioned that all or only a portion of shaft 28 may have alternate cross section configurations, such as, for example, oval, oblong, triangular, square, polygonal, irregular, uniform, non-uniform, offset, staggered, undulating, arcuate, variable and/or tapered. It is contemplated that the outer surface of shaft 28 may include one or a plurality of openings. It is further contemplated that all or only a portion of the outer surface of shaft 28 may have alternate surface configurations to enhance fixation with tissue such as, for example, rough, arcuate, undulating, mesh, porous, semi-porous, dimpled and/or textured according to the requirements of a particular application. It is envisioned that all or only a portion of shaft 28 may be cannulated.

In one embodiment, head 26 of bone screw 20 has a reduced thickness at a proximal end of head 26 and an increased thickness at a distal end of head 26 to enhance the low profile configuration of bone screw 20 with a body. As such, head 26 is tapered such that at least a portion of head 26 may seat more closely with the anatomy of a patient, thereby reducing the profile of bone screw 20. In one embodiment, at least a portion of an outer surface of head 26 is threaded for penetration with a body surface such that bone screw 20 has a low profile when fixed with a body of a patient. It is envisioned that the threads on the outer surface of head 26 may be continuous with the threads on shaft 28.

Connector 22 extends between a first end 32 and a second end 34. First end 32 includes a first disk 36 having a first splined surface 40. First splined surface 40 of first disk 36 is configured to mate with splines on a surface of receiver 24 to releasably fix receiver 24 with connector 22 in a selected rotatable position in a second plane, such as, for example, a transverse plane of the body relative to shaft 28 of bone screw 20. It is envisioned that receiver 24 may be coupled with connector 22 in alternate fixation configurations, such as, for example, friction fit, pressure fit, locking protrusion/recess, locking keyway and/or adhesive. It is further envisioned that receiver 24 may be disposed with connector 22 for relative movement thereto, such as, for example, transverse, perpendicular and/or other angular orientations such as acute or obtuse, co-axial and/or may be offset or staggered. It is contemplated that receiver 24 may move relative to connector 22 in alternate planes relative to a body, such as, for example, transverse and/or sagittal planes of a body. It is further contemplated that all or only a portion of first splined surface 40 of first disk 36 may have alternate surface configurations to enhance fixation with receiver 24, such as, for example, rough, arcuate, undulating, mesh, porous, semi-porous, dimpled and/or textured according to the requirements of a particular application.

First end 32 of connector 22 includes a first locking part, such as, for example, a circumferential flange 38 defining an opening 42 extending through first end 32 of connector 22 configured to receive a distal portion of receiver 24 to lock receiver 24 with connector 22. Opening 42 is round and is configured to receive a distal portion of receiver 24, which is at least partially arcuate. It is envisioned that all or only a portion of opening 42 may be variously configured and dimensioned, such as, for example, oval, oblong, square, rectangular, polygonal, irregular, uniform, non-uniform, offset, staggered, tapered, consistent or variable, depending on the requirements of a particular application. In one embodiment, opening 42 may extend through only a portion of first end 32 and not completely through. First splined surface 40 has a height, which is greater than the height of circumferential flange 38 such that first splined surface 40 is superior to circumferential flange 38.

Second end 34 of connector 22 includes a cylindrical outer surface comprising axial splines 44 circumferentially disposed about the outer surface of second end 34. Axial splines 44 are engageable with axial splines 31 disposed about the inner surface of first cavity 30 in head 26 for selective angular fixation of the bone screw 20 with connector 22. Axial splines 44 include a plurality of individual spline members that extend in parallel relation about the outer surface of second end 34. The configuration of axial splines 44 provides at least a portion of a mounting and alignment configuration for aligning and mounting bone screw 20 with connector 22 and receiver 24 during a surgical treatment. It is envisioned that the outer surface of second end 34 may have alternate cross section configurations, such as, for example, oval, oblong, triangular, rectangular, square, polygonal, irregular, uniform, non-uniform, variable and/or tapered. It is further envisioned that all or only a portion of the outer surface of second end 34 may have alternate surface configurations, such as, for example, rough, threaded for connection with other instruments, arcuate, undulating, porous, semi-porous, dimpled, polished and/or textured according to the requirements of a particular application.

In one embodiment, second end 34 of connector 22 includes a proximal portion 46 having a diameter, which is less than a diameter of a distal portion 48 of second end 34. The reduced diameter of proximal portion 46 facilitates assembly with bone screw 20 in that the reduced diameter more readily mates with first cavity 30 of bone screw 20. The reduced diameter of proximal portion 46 can be captured by first cavity 30 during manipulation of bone screw 20 by a practitioner. This configuration facilitates guiding of second end 34 of connector 22 within first cavity 30 for mating of axial splines 31 on the interior surface of first cavity 30 with axial splines 44 on the exterior surface of second end 34 of connector 22 in relative rotatable alignment. Splines 31 are selectively aligned with splines 44 according to the requirements of a particular application. It is envisioned that distal portion 48 may have a uniform, pointed, planar or beveled configuration. It is further envisioned that proximal portion 46 may have a diameter, which is equal to a diameter of a distal portion 48 of second end 34.

Second end 34 of connector 22 is configured for disposal within first cavity 30 of bone screw 20 along axis A₁ in the coronal plane of the body for selective fixation in a position along axis A₁. Second end 34 of connector 22 is moveable along axis A₁ relative to shaft 28 of bone screw 20. It is envisioned that second end 34 may be inserted into first cavity 30. Second end 34 is moved within first cavity 30 in the coronal plane along axis A₁, in a first axial direction or a second axial direction oriented in an opposing direction to the first axial direction, by sliding second end 34 relative to first cavity 30. First cavity 30 is engageable with second end 34 to align the component parts in relative rotatable alignment in angular increments about their relative circumferential surfaces. The angular increments correspond to a spline teeth angle of splines 31, 44. It is contemplated that the spline teeth angle may be in a range of approximately 5 to 20 degrees.

Receiver 24 is attachable to first end 32 of connector 22 and is selectively rotatable in the transverse plane of the body and selectively fixable in a position within the transverse plane. Receiver 24 includes a body portion having an implant cavity 56 defining a second axis A₂. Implant cavity 56 is configured to receive and movably support at least a portion of an implant, such as, for example, a vertebral rod, such that the implant can translate axially within implant cavity 56 along second axis A₂. Implant cavity 56 is rotatable about a third axis A₃ disposed transverse to second axis A₂ such that an implant disposed in implant cavity 56 and posterior to first end 32 of connector 22 is rotatable in a first plane, such as, for example, a coronal plane of the body, relative to first end 32 of connector 22 in a configuration for selective fixation with first end 32 of connector 22. It is contemplated that at least a portion of the implant may be disposed within implant cavity 56 for relative movement in orientations relative to second axis A₂, such as, for example, transverse, perpendicular and/or other angular orientations such as acute or obtuse, co-axial and/or may be offset or staggered. It is further contemplated that at least a portion of the implant may be disposed within implant cavity 56 in alternate planes relative to a body, such as, for example, coronal and/or sagittal planes of a body.

Implant cavity 56 is substantially tubular and is configured to receive at least a portion of a cylindrical implant, such as, for example, a vertebral rod. It is envisioned that implant cavity 56 may be variously configured and dimensioned, such as, for example, round, oval, oblong, square, rectangular, polygonal, irregular, uniform, non-uniform, offset, staggered, tapered, consistent or variable, depending on the requirements of a particular application. It is further envisioned that implant cavity 56 may be movable relative to first axis A₁ in orientations, such as, for example, transverse, perpendicular and/or other angular orientations such as acute or obtuse, co-axial and/or may be offset or staggered. It is contemplated that implant cavity 56 may move relative to connector 22 in alternate planes relative to a body, such as, for example, transverse and/or sagittal planes of a body. In one embodiment, implant cavity 56 may be disposed at an angle of about 30 to about 150 degrees relative to first cavity 30 of bone screw 20 and second axis A₂ may be disposed at an angle of about 30 to about 150 degrees relative to first axis A₁.

Receiver 24 includes a threaded cavity 58 extending along third axis A₃ disposed transverse to second axis A₂ configured to receive a coupling member, such as, for example, a setscrew to releasably fix an implant within implant cavity 56 of receiver 24 in a selected position at an angle relative to second axis A₂. The setscrew is threaded with cavity 58 into fixed engagement with an outer surface of the implant. It is envisioned that the implant may be coupled with receiver 24 in alternate fixation configurations, such as, for example, friction fit, pressure fit, locking protrusion/recess, locking keyway and/or adhesive. It is contemplated that all or only a portion of receiver 24 may have alternate surface configurations to enhance fixation with the implant, such as, for example, rough, arcuate, undulating, mesh, porous, semi-porous, dimpled and/or textured according to the requirements of a particular application.

Receiver 24 includes a first locking part, such as, for example, a distal portion 60 configured for disposal within opening 42 of connector to connect receiver 24 with connector 22. Distal portion 60 includes a first prong, such as, for example, a first arm 62, a second prong, such as, for example, a second arm 64 and a wishbone part 66. First and second arms 64, 66 extend perpendicularly from a bottom surface of the body portion receiver 24. Distal portion 60 of receiver 24 has a reduced thickness configuration for disposal in opening 42 in first disk 46 to rotatably connect receiver 24 with connector 22. In one embodiment, receiver 24 (and implant cavity 56 extending through receiver 24) may be rotated through an angle of 0 to 360 degrees relative to connector 22. First and second arms 64, 66 include flanges 68, 70 extending laterally from an end of first and second arms 64, 66 opposite the end of first and second arms 64, 66 extending from the bottom surface of receiver 24. Flanges 68, 70 engage a second locking part, such as, for example, an interior surface of opening 42 when distal portion 60 of receiver 24 is inserted into opening 42 of connector 22. It is envisioned that first and second arms 64, 66 can be squeezed together either by external pressure, snap-fit, friction fit and/or threaded engagement to facilitate insertion of distal portion 60 into opening 42. In one embodiment, the body portion of receiver 24 has an elastic configuration such that first and second arms 64, 66 are resiliently biased outwardly. It is envisioned that distal portion 60 may be spring-loaded, or include a biased member, or a shape-memory member.

Distal portion 60 of receiver 24 is fixed with connector 22 and relative rotation thereto with wishbone part 66. Wishbone part 66 has base and extending lobes and is disposable to engage and force first and second arms 64, 66 into fixation with opening 42 in circumferential flange 38 to lock receiver 24 with connector 22. After first and second arms 64, 66 of distal portion 60 are inserted within opening 42 in first disk 46 of connector 22 wishbone part 66 may be disposed between first and second arms 64, 66. Flanges 68, 70 of first and second arms 64, 66 engage the inner surface of opening 42 such that first and second arms 64, 66 retain receiver 24 with connector 22. Wishbone part 66 occupies some or all of the space between first and second arms 64, 66 to prevent first and second arms 64, 66 from inward movement to prevent release of flanges 68, 70. This configuration prevents undesired removal of distal portion 60 of receiver 24 from connector 22. In one embodiment, wishbone part 66 may have a width that is approximately equal to or slightly greater than the gap between first and second arms 64, 66 when first and second arms 64, 66 and receiver 24 are in an unstressed state. In one embodiment, wishbone part 66 may include a block, such as, for example, a ball, cylinder, planar solid or other relatively solid structure.

Receiver 24 includes a second disk 50 having a first face defining a second splined surface 52 defined by axial splines 54 that mate with the splines on first splined surface 40 of first disk 36 to releasably fix receiver 24 with connector 22 in a selected rotatable position in a plane, such as, for example, a coronal plane of the body relative to shaft 28 of bone screw 20. It is contemplated that all or only a portion of second splined surface 52 may have alternate surface configurations to enhance fixation with connector 22, such as, for example, rough, arcuate, undulating, mesh, porous, semi-porous, dimpled and/or textured according to the requirements of a particular application. Splines 54 and the splines on first splined surface 40 of first disk 36 are configured to mesh such that receiver 24 (including implant cavity 56) and second disk 50 can rotate and lock at different angles relative to connector 22 in the transverse plane such that implant cavity 56 may rotate about third axis A₃ such that a vertebral rod disposed in implant cavity 56 and posterior to first end 32 of connector 22 is selectively rotatable in a coronal plane of a body relative to first end 32 of connector 22 in a configuration for selective angular fixation with first end 32 of connector 22. It is envisioned that implant cavity 56 (or an implant received within implant cavity 56) may be rotated through an angle of 0 to 360 degrees relative to connector 22. Second disk 50 is locked in position relative to first disk 36 by forcing disks 36, 50 into engagement. In one embodiment, disks 36, 50 are resiliently biased towards for fixed engagement.

Second disk 50 includes a second face, such as, for example, an upper surface 72 opposite second splined surface 52 that defines an opening 74 extending through second disk 50. Opening 74 is configured for disposal of distal portion 60 of receiver 24. Upper surface 72 of second disk 50 defines a channel 76 having approximately the same diameter as an implant disposed in implant cavity 56. It is envisioned that channel 76 may also have a V-shaped cross section or other configuration. Upper surface 72 of second disk 50 further includes one or more lateral openings 78 to accommodate a portion of the body portion of receiver 24 such that second disk 50 can slide onto receiver 24 so that second disk 50 fits relatively closely and/or snugly against receiver 24.

In assembly, operation and use, the implant system including bone screw 20, connector 22 and receiver 24, similar to that described above with regard to FIGS. 1 and 2, is employed with a surgical procedure for treatment of a spinal disorder affecting a section of a spine and/or ilium bones of a pelvis of a patient, as discussed herein. The implant system may also be employed with other surgical procedures. The implant system is employed with a surgical procedure for treatment of a condition or injury of an affected section of the spine including vertebrae V, which may include sacrum S, and/or ilium I, as shown in FIG. 3. It is contemplated that the implant system including bone screw 20, connector 22 and receiver 24 is attached to vertebrae V and/or ilium I for a surgical arthrodesis procedure, such as fusion, and/or dynamic stabilization application of the affected section of the spine to facilitate healing and therapeutic treatment.

In use, to treat the affected section of the spine and/or ilium bones of a pelvis, a medical practitioner obtains access to a surgical site including vertebra V and/or ilium I in any appropriate manner, such as through incision and retraction of tissues. It is envisioned that the implant system including bone screw 20, connector 22 and receiver 24 may be used in any existing surgical method or technique including open surgery, mini-open surgery, minimally invasive surgery and percutaneous surgical implantation, whereby the vertebrae V and/or ilium I is accessed through a micro-incision, or sleeve that provides a protected passageway to the area. Once access to the surgical site is obtained, the particular surgical procedure is performed for treating the bone disorder. The implant system including bone screw 20, connector 22 and receiver 24 is then employed to augment the surgical treatment. The implant system including bone screw 20, connector 22 and receiver 24 can be delivered or implanted as a pre-assembled device or can be assembled in situ. The implant system may be completely or partially revised, removed or replaced.

In one embodiment, the implant system includes one or a plurality of vertebral rods 108, bone fasteners 20, connectors 22 and receivers 24 for attaching rods 108 to vertebrae V, as shown in FIG. 3. The implant system of the present disclosure extends from a first portion 104 to a second portion 106 disposed adjacent a sacroiliac region (SIR) of the patient. Second portion 106 includes two axially aligned and spaced apart rods 108. Rods 108 each have a rigid, arcuate portion 110 extending across a sacrum S and ilium I of region SIR. A first bone fastener 20 is configured for fixation with an ilium surface I1 and a second bone fastener 20 is configured for fixation with an ilium surface I2. Pilot holes are made in ilium surfaces I1, I2 for receiving first and second bone screws 20. Each threaded shaft 28 of first and second bone screws 20 are inserted or otherwise connected to ilium surfaces I1, I2, according to the particular requirements of the surgical treatment. Connector 22 is attached with bone screw 20, and receiver 24 is attached with connector 22, as described above.

According to the orientation and position of each arcuate portion 110, connector 22 is aligned in relative rotatable alignment with first cavity 30 of bone screw 20. Second end 34 of connector 22 is configured for disposal within first cavity 30 of bone screw 20 along axis A₁ in the coronal plane of the body for selective fixation in a position along axis A₁. Second end 34 is moved within first cavity 30 in the coronal plane along axis A₁, in a first axial direction or a second axial direction oriented in an opposing direction to the first axial direction according to the orientation and position of each arcuate portion 110, by sliding second end 34 relative to first cavity 30.

Each receiver 24 may be rotated relative to each connector 22 such that arcuate portion 110 of each rod 108 may be moved within implant cavity 56 of receiver 24 by sliding arcuate portion 110 within implant cavity 56 such that each rod 108 is posterior to first end 32 of connector 22. Implant cavity 56 is selectively rotated relative to third axis A₃ in a first plane, such as, for example, the coronal plane of the patient corresponding to the orientation and position of each arcuate portion 110. This configuration allows orientation of implant cavity 56 to receive each arcuate portion 110 for disposal of each arcuate portion 110 therein. A setscrew may be threaded with threaded cavity 58 of receiver 24 into fixed engagement with an outer surface of each arcuate portion 110 to fix receiver 24 in the selected rotation orientation. The splined surfaces of disks 36, 50 are brought into fixed engagement, as discussed above, to lock receiver 24 and connector 22 in the selected rotation orientation. It is contemplated that the implant system configuration of bone screw 20, connector 22 and receiver 24 allows the implant system to be disposed close to a body surface, so as to be low profile, while retaining adjustability and maintaining strength of fixation and/or attachment with the body surface.

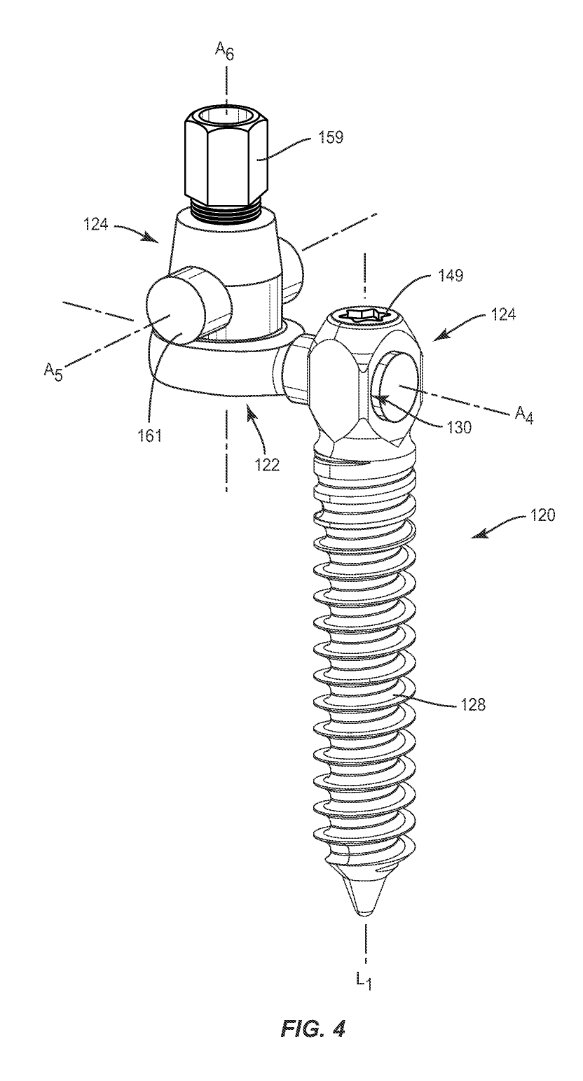

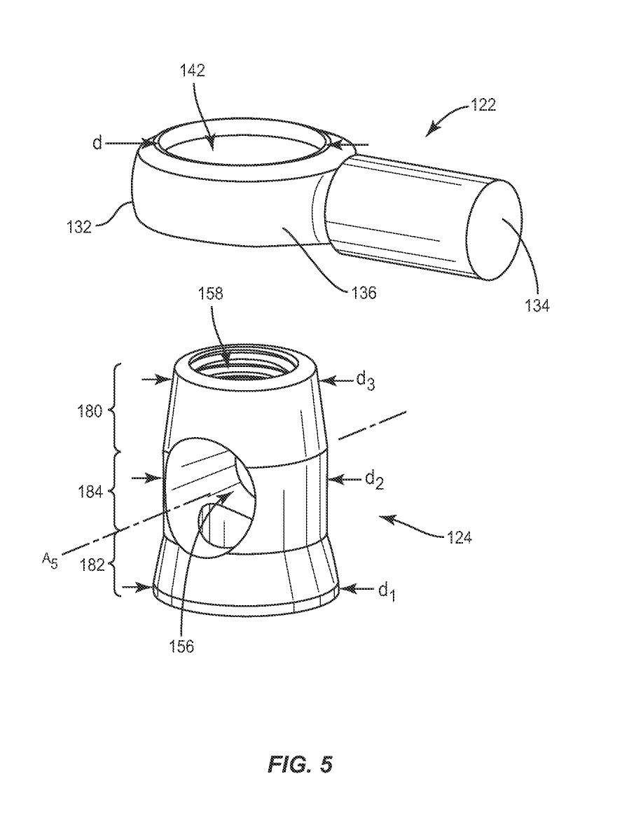

In one embodiment, as shown in FIGS. 4 and 5, the implant system includes a bone screw 120, a connector 122 and a receiver 124, similar to the components described above with regard to FIGS. 1-3. Bone screw 120 includes a proximal head 126 and a distal threaded shaft 128 configured to penetrate bone. Head 126 includes an inner surface that defines a first cavity 130 (not shown) that defines an axis A₄. First cavity 130 is configured to receive and movably support at least a portion of connector 122 such that connector 122 can translate axially within first cavity 130 along axis A₄ through a plane, such as, for example, a coronal plane of a body. First cavity 130 has a tubular configuration for receiving a corresponding round portion of connector 122 and extends through head 126 of bone screw 120 so as to form a passageway through bone screw 120. First cavity 130 is smooth and continuous such that first cavity 130 is free of any gaps or protrusions. Distal threaded shaft 128 defines a longitudinal axis L₁ disposed transverse to axis A₄.

Connector 122 extends between a first end 132 and a cylindrical second end 134. First end 132 of connector 122 includes a first disk 136 defining an opening 142 configured for engagement with receiver 124 to retain receiver 124 with connector 122. Opening 142 has a diameter d and extends through first disk 136 of connector 122 so as to define a passageway extending perpendicularly through first disk 136. At least a portion of an outer surface of receiver 124 interfaces at least a portion of an inner surface of opening 142 to releasably fix receiver 124 with connector 122 in a selected rotatable position in a plane, such as, for example, a coronal plane of the body relative to shaft 128 of bone screw 120.

Second end 134 has a cylindrical outer surface, which is smooth, and continuous such that second end 134 is free of gaps or protrusions. Second end 134 has a diameter, which is less than that of first cavity 130 such that second end 134 may be received within first cavity 130 of bone screw 120 to retain connector 122 with bone screw 120. Second end 134 of connector 122 is configured for movement within first cavity 130 of bone screw 120 along axis A₄ for selective fixation in a position along axis A₄. Second end 134 of connector 122 is moveable along axis A₄ relative to shaft 128 of bone screw 120. It is envisioned that second end 134 may be inserted into first cavity 130 and moved within first cavity 130 along axis A₄, in a first axial direction or a second axial direction oriented in an opposing direction to the first axial direction, by sliding second end 134 relative to first cavity 130. First cavity 130 is engageable with second end 134 to align the component parts in relative rotatable alignment.

Head 126 of bone screw 120 includes a first threaded cavity 147 (not shown) configured to receive a coupling member, such as, for example, a setscrew 149 to releasably fix connector 122 with bone screw 120 in a selected position. Setscrew 149 is threaded with first threaded cavity 147 into fixed engagement with an outer surface of second end 134 of connector 122.

Receiver 124 is attachable to first end 132 of connector 122 and is selectively rotatable in the transverse plane of the body and selectively fixable in a position within the transverse plane. Receiver 124 includes a body defined by a proximal portion 180, a distal portion 182 and a medial portion 184 between proximal and distal portions 180, 182. Distal portion 182 has a diameter d₁, which is greater than diameter d of opening 142 in connector 122. Medial portion 184 has a diameter d₂, which is less than diameter d of opening 142, and proximal portion 180 has a diameter d₃, which is less than diameter d₂ of medial portion 184. Receiver 124 is tapered between distal and proximal portions 182, 180 such that receiver 124 may be inserted into opening 142 of first disk 136 of connector 122 leading with proximal portion 180 of receiver 124. Receiver 124 may then be advanced within opening 142 until the inner surface of opening 142 engages an outer surface of distal portion 182 such that the inner surface of opening 142 and the outer surface of distal portion 182 are in close fitting engagement with one another. Since diameter d₁ of distal portion 182 is greater than diameter d of opening 142, connector 122 is prevented from moving past distal portion 182.

The body of receiver 124 includes an implant cavity 156 defining an axis A₅. Implant cavity 156 is configured to receive and movably support at least a portion of an implant, such as, for example, a vertebral rod 161, such that rod 161 can translate axially within implant cavity 156 along axis A₅. Implant cavity 156 is rotatable about an axis A₆ disposed transverse to axis A₅ such that rod 161 disposed in implant cavity 156 and posterior to first end 132 of connector 122 is rotatable in a first plane, such as, for example, a coronal plane of the body, relative to first end 132 of connector 122 in a configuration for selective fixation with first end 132 of connector 122.

Receiver 124 includes a second threaded cavity 158 extending along axis A₆ disposed transverse to axis A₄ configured to receive a coupling member, such as, for example, a setscrew 159 to releasably fix an implant, such as for example, rod 161 with receiver 124. Setscrew 159 is threaded with second threaded cavity 158 into fixed engagement with an outer surface of rod 161.

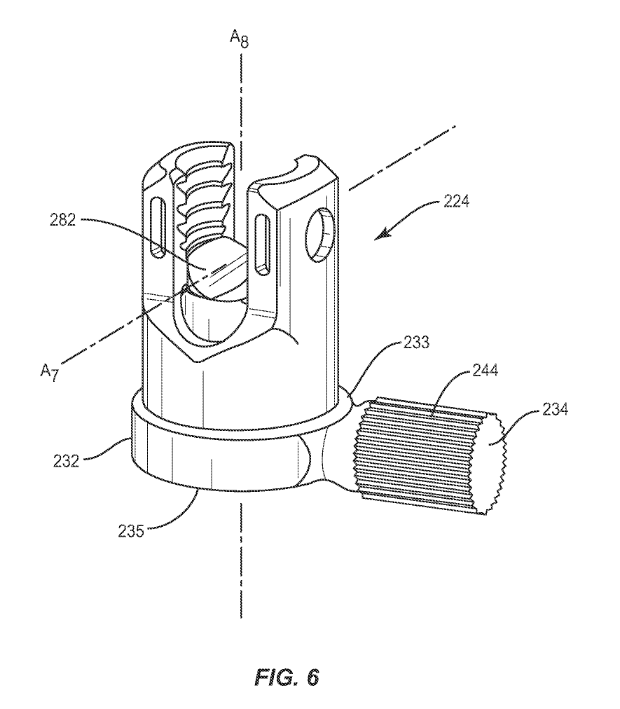

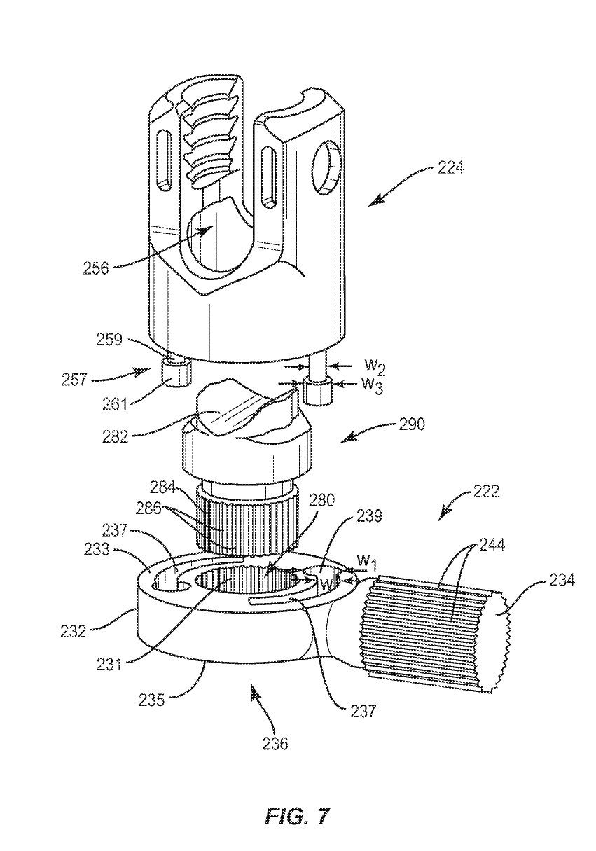

In one embodiment, as shown in FIGS. 6 and 7, the implant system includes bone screw (not shown), a connector 222 and a receiver 224, similar to the components described above. Connector 222 extends between a first end 232 and a second end 234. First end 232 includes a first disk 236 having an upper surface 233 and a lower surface 235. Upper surface 233 includes at least one keyway 237 configured for engagement with a key extending from receiver 224. As shown in FIG. 7, the implant system includes two (2) keyways 237. Each keyway 237 defines a first arcuate cavity having a width w and an opening 239 having a width w₁, which is greater than width w. Keyway 237 also defines a second arcuate cavity having a width, which is greater than the first arcuate cavity positioned within connector 222 distal to the first arcuate cavity. A key extending from receiver 224 having a width which is less than width w₁ but greater than width w is inserted into opening 239 and advanced distally from the first arcuate cavity of keyway 237 into the second arcuate cavity. The key may then be advanced within keyway 237 by rotating receiver 224 relative to connector 22. The reduced width of the key relative to the first arcuate cavity of keyway 237 prevents the key from being removed from the first arcuate cavity of keyway 237 until the key is aligned with opening 239. To remove receiver 224 from connector 222, receiver 224 is rotated relative to connector 222 such that the keys extending from receiver 224 are aligned with openings 239 in connector 222. Connector 222 may then be removed from receiver 224 by removing the keys extending from receiver 224 from keyway 237 through openings 239.

First disk 236 includes an inner surface that defines a first cavity 280. First cavity 280 is configured to receive and movably support at least a portion of a saddle such that the saddle can translate axially within first cavity 280. First cavity 280 has a tubular configuration for receiving a corresponding round portion of the saddle and extends through first disk 236 so as to form a passageway through first disk 236. It is envisioned that all or only a portion of first cavity 280 may be variously configured and dimensioned, such as, for example, oval, oblong, square, rectangular, polygonal, irregular, uniform, non-uniform, offset, staggered, tapered, consistent or variable, depending on the requirements of a particular application. In one embodiment, first cavity 280 may extend through only a portion of first disk 236 and not completely through, such that, for example, first cavity 280 extends through upper surface 233 but not through lower surface 235 of first disk 236.

First cavity 280 includes axial splines 231 circumferentially disposed about the inner surface of first disk 236. First cavity 280 provides at least a portion of a mounting and alignment configuration for mating connector 222 with the saddle and receiver 224 during a surgical treatment. Axial splines 231 in first cavity 280 include a plurality of individual spline members that extend in parallel relation about first cavity 280. Axial splines 231 in first cavity 280 mate with splines on the saddle to align connector 222 with the saddle and/or receiver 224.

Second end 234 of connector 222 includes a cylindrical outer surface comprising axial splines 244 circumferentially disposed about the outer surface of second end 234. Axial splines 244 are engageable with axial splines disposed about a cavity of a head of a bone screw, similar to bone screw 30, for selective angular fixation of the bone screw with connector 222. Axial splines 244 include a plurality of individual spline members that extend in parallel relation about the outer surface of second end 234. The configuration of axial splines 244 provides at least a portion of a mounting and alignment configuration for aligning and mounting the bone screw with connector 222 and receiver 224 during a surgical treatment.

Second end 234 of connector 222 is configured for movement within the cavity of the bone screw along an axis in the coronal plane of the body for selective fixation in a position along the axis. Second end 234 of connector 222 is moveable along the axis relative to a shaft of the bone screw. Second end 234 is moved within the cavity of the bone screw in the coronal plane along the axis, in a first axial direction or a second axial direction oriented in an opposing direction to the first axial direction, by sliding second end 234 relative to the cavity. The cavity is engageable with second end 234 to align the component parts in relative rotatable alignment.

Receiver 224 is attachable to first end 232 of connector 222 and is selectively rotatable in the transverse plane of the body and selectively fixable in a position within the transverse plane. Receiver 224 includes a body portion having an implant cavity 256. Implant cavity 256 is configured to receive and movably support at least a portion of an implant, such as, for example, a vertebral rod, such that the implant can translate axially within implant cavity 256. Implant cavity 256 defines an axis A₇ and is rotatable about an axis A₈ disposed transverse to axis A₇ such that an implant disposed in implant cavity 256 and posterior to first end 232 of connector 222 is rotatable in a first plane, such as, for example, a coronal plane of the body, relative to first end 232 of connector 222 in a configuration for selective fixation with first end 232 of connector 222.

The body portion of receiver 224 includes at least one key 257 defined by a first post 259 and a second post 261 extending perpendicularly from first post 259. Each key 257 is configured for engagement with a keyway 237. Keys 257 extend perpendicularly from a bottom surface of the body portion of receiver 224. First post 259 has a width w₂ which is less than width w₁ of opening 239 and width w of keyway 237. Second post 261 has a width w₃ which is less than width w₁ of opening 239 but greater than width w of keyway 237 such that second post 261 may be inserted into opening 239.

Key 257 is inserted into opening 239 and advanced distally from the first arcuate cavity of keyway 237 into the second arcuate cavity. Key 257 may then be advanced within keyway 237 by rotating receiver 224 relative to connector 222. The reduced width of key 257 relative to the first arcuate cavity of keyway 237 prevents key 257 from being removed from the first arcuate cavity of keyway 237 until key 257 is aligned with opening 239. To remove receiver 224 from connector 222, receiver 224 is rotated relative to connector 222 such that key 257 extending from receiver 224 is aligned with opening 239 in connector 222. Connector 222 may then be removed from receiver 224 by removing key 257 from keyway 237 through openings 239.

The bottom surface of the body portion of receiver 224 includes an opening extending therethrough configured to receive a saddle 290 including a top portion 282 and a bottom portion 284. Top portion 282 of saddle 290 is concavely curved and is configured to support an implant, such as, for example, a vertebral rod, positioned within implant cavity 256 of receiver 224. In one embodiment, the concavely curved portion of saddle 290 is continuous with a portion of receiver 224 defining implant cavity 256 such that there are no gaps or protrusions between the concavely curved portion of saddle 290 and the portion of receiver 224 defining implant cavity 256 when saddle 290 is inserted into the opening in the bottom surface of the bottom portion of receiver 224. It is envisioned that the concavely curved portion of saddle 290 may be positioned superior to a portion of receiver 224 defining implant cavity 256 when saddle 290 is connected with receiver 224.

Bottom portion 284 of saddle 290 includes a cylindrical outer surface comprising axial splines 286 circumferentially disposed about the outer surface of bottom portion 284. Axial splines 286 are engageable with axial splines 231 disposed about the inner surface of first disk 236 for selective angular fixation of the connector 222 with saddle 290. Axial splines 286 include a plurality of individual spline members that extend in parallel relation about the outer surface of bottom portion 284. The configuration of axial splines 286 provides at least a portion of a mounting and alignment configuration for aligning and mounting connector 222 with saddle 290 and receiver 224 during a surgical treatment.

Bottom portion 284 of saddle 290 is configured for movement within first cavity 280 of connector 222. Second end 234 of connector 222 is moveable along the axis relative to the shaft of the bone screw. It is envisioned that bottom portion 284 may be inserted into first cavity 280. First cavity 280 of connector 22 is engageable with bottom portion 284 of saddle 290 to align the component parts in relative rotatable alignment.

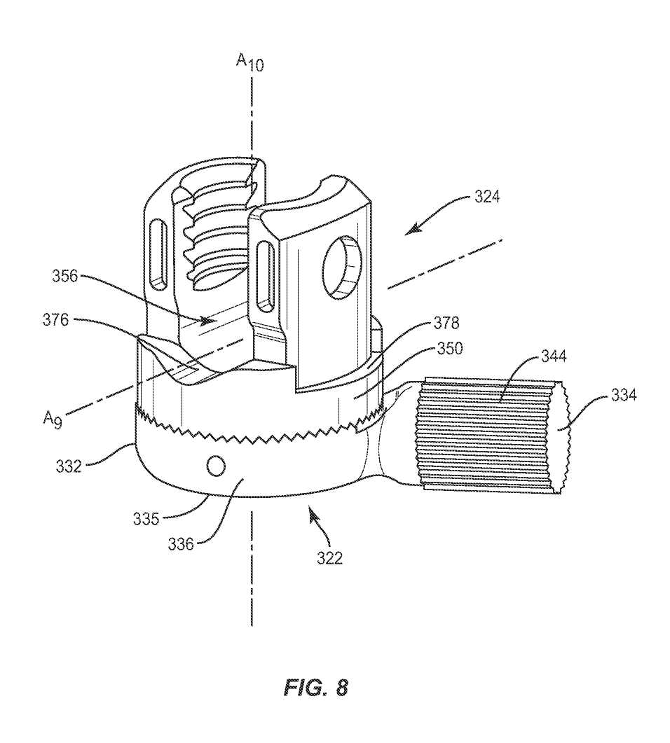

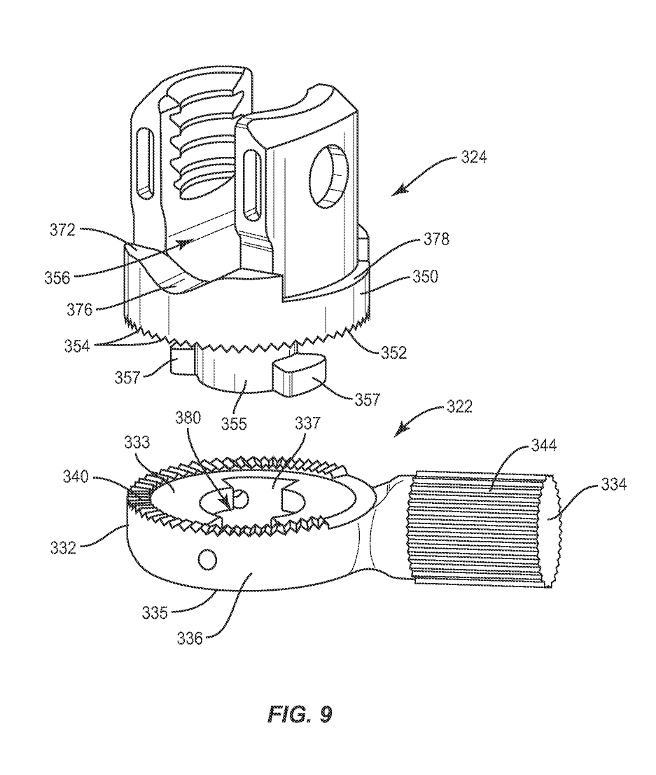

In one embodiment, as shown in FIGS. 8 and 9, the implant system includes bone screw 20 described above with regard to FIGS. 1-3, a connector 322 and a receiver 324, similar to the components described above. Connector 322 extends between a first end 332 and a second end 334. First end 332 includes a first disk 336 having an upper surface 333 and a lower surface 335. First disk 336 includes an inner surface that defines a first cavity 380. First cavity 380 is configured to receive and movably support at least a portion of receiver 224 such that receiver 324 can translate axially within first cavity 380. First cavity 380 has a round configuration for receiving a corresponding round portion of receiver 324 and extends through first disk 336 so as to form a channel through first disk 336. In one embodiment, first cavity 380 may extend through only a portion of first disk 336 and not completely through, such that, for example, first cavity 380 extends through upper surface 333 but not through lower surface 335 of first disk 336.

Upper surface 333 of first disk 336 includes a pair of spaced apart arcuate keyways 337 extending from opposite sides of first cavity 380 configured for engagement with a pair of spaced apart keys extending from receiver 324. Keyways 337 each include an arcuate portion between angled walls, which extend from first cavity 380. Keyways 337 each define a first passageway. A second passageway 339 (not shown) is disposed in upper surface 333 of first disk 336 distal to keyways 337. Second passageway 339 is in communication with keyways 337 and has a transverse orientation relative to keyways 337. A key extending from receiver 324 is first inserted into a keyway 337. The key is then advanced distally out of keyway 337 and into second passageway 339. The key may be rotated within second passageway 339 to connect receiver 324 with connector 322.

Upper surface 333 of first disk 336 includes a first splined surface 340. First splined surface 340 of first disk 336 is configured to mate with splines on a surface of receiver 324 to releasably fix receiver 324 with connector 322 in a selected rotatable position in a plane, such as, for example, a coronal plane of the body relative to shaft 28 of bone screw 20.

Second end 334 of connector 322 includes a cylindrical outer surface comprising axial splines 344 circumferentially disposed about the outer surface of second end 334. Axial splines 344 are engageable with axial splines 31 disposed about first cavity 30 of head 26 for selective angular fixation of the bone screw 20 with connector 322. Axial splines 344 include a plurality of individual spline members that extend in parallel relation about the outer surface of second end 334. The configuration of axial splines 344 provides at least a portion of a mounting and alignment configuration for aligning and mounting bone screw 20 with connector 322 and receiver 324 during a surgical treatment.

Second end 334 of connector 322 is configured for movement within first cavity 30 of bone screw 20 along axis A₁ in the coronal plane of the body for selective fixation in a position along axis A₁. Second end 334 of connector 322 is moveable along axis A₁ relative to shaft 28 of bone screw 20. It is envisioned that second end 334 may be inserted into first cavity 30. Second end 334 is moved within first cavity 30 in the coronal plane along axis A₁, in a first axial direction or a second axial direction oriented in an opposing direction to the first axial direction, by sliding second end 334 relative to first cavity 30. First cavity 30 is engageable with second end 334 to align the component parts in relative rotatable alignment.

Receiver 324 is attachable to first end 332 of connector 322 and is selectively rotatable in the transverse plane of the body and selectively fixable in a position within the transverse plane. Receiver 324 includes a body portion having an implant cavity 356. Implant cavity 356 is configured to receive and movably support at least a portion of an implant, such as, for example, a vertebral rod, such that the implant can translate axially within implant cavity 356. Implant cavity 356 defines an axis A₉ and is rotatable about an axis A₁₀ disposed transverse to axis A₉ such that an implant disposed in implant cavity 356 and posterior to first end 332 of connector 322 is rotatable in a plane, such as, for example, a coronal plane of the body, relative to first end 332 of connector 322 in a configuration for selective fixation with first end 332 of connector 322.

The body portion of receiver 324 includes a cylindrical distal portion 355 having a diameter which is less than that of first cavity 380 in connector 322 such that distal portion 344 may be received within first cavity 380. Receiver 324 also includes a pair of spaced apart keys 357 configured for engagement with keyways 337 extending from distal portion 355. Keys 357 extend transversely from distal portion 355 of receiver 324. Keys 357 are inserted into keyways 337 of connector 322 to connect receiver 324 with receiver 324. Keys 357 are then advanced distally through keyways 337 and into second passageway 339. Keys 357 may then be rotated within second passageway 339 to connect receiver 324 with connector 322. It is envisioned that keys 357 may be rotated between −135 and 135 degrees relative to keyways 337 to connect receiver 324 with connector 322.

Receiver 324 includes a second disk 350 having a first face defining a second splined surface 352 defined by axial splines 354 that mate with the splines on first splined surface 340 of first disk 336 to releasably fix receiver 324 with connector 322 in a selected rotatable position in a plane, such as, for example, a coronal plane of the body relative to shaft 28 of bone screw 20. Splines 354 on second disk 350 and the splines on first splined surface 340 of first disk 336 are configured to mesh such that receiver 324 (including implant cavity 356 and second disk 350) can rotate and lock at different angles relative to connector 322 such that a vertebral rod disposed in implant cavity 356 and posterior to first end 332 of connector 322 is selectively rotatable in a coronal plane of a body relative to first end 332 of connector 322 in a configuration for selective angular fixation with first end 332 of connector 322. It is envisioned that implant cavity 356 (or an implant received within implant cavity 356) may be rotated through an angle of 0 to 360 degrees relative to connector 322. Second disk 350 is locked in position relative to first disk 336 by forcing disks 336, 350 into engagement.

Second disk 350 includes a second face, such as, for example, an upper surface 372 opposite second splined surface 352 that defines an opening 374 (not shown) extending through second disk 350. Opening 374 is configured for disposal of distal portion 355 of receiver 324. Upper surface 372 of second disk 350 includes a channel 376 having approximately the same diameter as an implant disposed in implant cavity 356. Second disk 350 includes one or more lateral openings 378 to accommodate a portion of the body portion of receiver 324 such that second disk 350 can slide onto receiver 324 so that second disk 350 fits relatively closely and/or snugly against receiver 324.

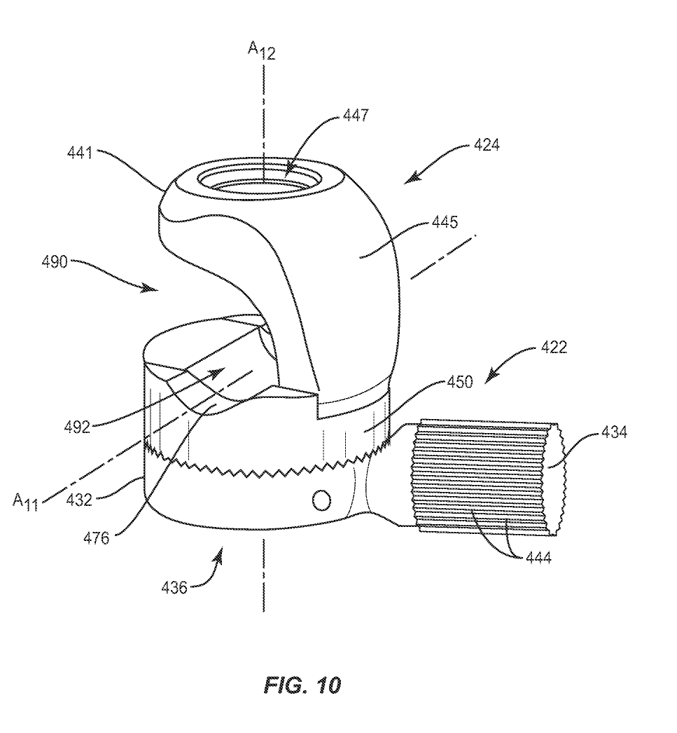

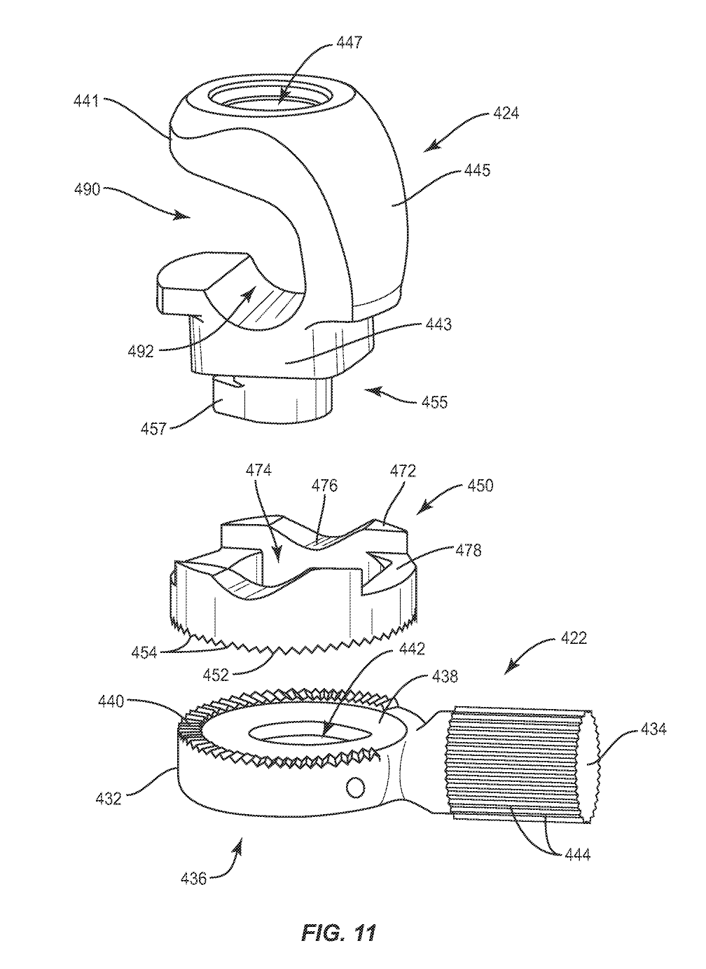

In one embodiment, as shown in FIGS. 10 and 11, the implant system includes bone screw 20 described above, a connector 422 and a receiver 424, similar to the components described above. Connector 422 extends between a first end 432 and a second end 434. First end 432 includes a first disk 436 having a first splined surface 440. First splined surface 440 of first disk 436 is configured to mate with splines on a surface of receiver 424 to releasably fix receiver 424 with connector 422 in a selected rotatable position in a plane, such as, for example, a coronal plane of the body relative to shaft 28 of bone screw 20.

First end 432 of connector 422 includes a first locking part comprising a circumferential flange 438 defining a keyway 442 configured for engagement with a distal portion of receiver 424 such that the distal portion of receiver 424 is lockable with keyway 442 to retain receiver 424 with connector 422. Keyway 442 defines a first passageway. A second passageway 462 (not shown) is disposed in first disk 436 distal to keyway 442. Second passageway 462 is in communication with keyway 442 and has a diameter, which is greater than that of keyway 442. A key extending from receiver 424 is first inserted into keyway 442. The key is then advanced distally out of keyway 442 and into second passageway 462. The key may be rotated within second passageway 462 to connect receiver 424 with connector 422. First splined surface 440 has a height, which is greater than the height of circumferential flange 438 such that first splined surface 440 is superior to circumferential flange 438.

Second end 434 of connector 422 includes a cylindrical outer surface comprising axial splines 444 circumferentially disposed about the outer surface of second end 434. Axial splines 444 are engageable with axial splines 31 disposed about the inner surface of head 26 for selective angular fixation of the bone screw 20 with connector 422. Axial splines 444 include a plurality of individual spline members that extend in parallel relation about the outer surface of second end 434. The configuration of axial splines 444 provides at least a portion of a mounting and alignment configuration for aligning and mounting bone screw 20 with connector 422 and receiver 424 during a surgical treatment.

Second end 434 of connector 422 is configured for movement within first cavity 30 of bone screw 20 along axis A₁ in the coronal plane of the body for selective fixation in a position along axis A₁. Second end 434 of connector 422 is moveable along axis A₁ relative to shaft 28 of bone screw 20. It is envisioned that second end 434 may be inserted into first cavity 30. Second end 434 is moved within first cavity 30 in the coronal plane along axis A₁, in a first axial direction or a second axial direction oriented in an opposing direction to the first axial direction, by sliding second end 434 relative to first cavity 30. First cavity 30 is engageable with second end 434 to align the component parts in relative rotatable alignment.

Receiver 424 is attachable to first end 432 of connector 422 and is selectively rotatable in the transverse plane of the body and selectively fixable in a position within the transverse plane. Receiver 424 includes a body portion defining a receiving portion which is substantially C-shaped and includes an upper leg 441, a lower leg 443 and an intermediate portion 445 joining upper leg 441 and lower leg 443. Upper leg 441 has an aperture 447 into which a setscrew can be threaded. Aperture 447 may have a longitudinal axis that is perpendicular to upper leg 441, or such axis may be angled with respect to upper leg 441, e.g. toward intermediate portion 445. Intermediate portion 445 joins legs 441, 443. Taken together, legs 441, 443 and intermediate portion 445 form substantially a C-shape. The receiving portion of receiver 424 defines a posterior opening 490 and a lateral opening 492, both configured to configured to receive and movably support at least a portion of an implant, such as, for example, a vertebral rod. Lateral opening 492 defines a concavely curved bottom surface and is configured such that the implant can translate axially within lateral opening 492. Posterior opening 490 is configured such that the implant can translate transversely within posterior opening 490 for disposal in lateral opening 492. Lateral opening 492 defines an axis A₁₁ and is rotatable about an axis A₁₂ disposed transverse to axis A₁₁ such that an implant disposed in lateral opening 492 and posterior to first end 432 of connector 422 is rotatable in a plane, such as, for example, a coronal plane of the body, relative to first end 432 of connector 422 in a configuration for selective fixation with first end 432 of connector 422.

The body portion of receiver 424 includes a cylindrical distal portion 455 having a flange 457 extending from an end of distal portion 455 configured to be received within keyway 442 of circumferential flange 438 such distal portion 455 is lockable with keyway 442 to retain receiver 424 with connector 422. Distal portion 455 extends transversely from a bottom surface of receiver 424. Distal portion 455 is inserted into keyway 442 in connector 422 to connect receiver 424 with connector 422. Distal portion 455 may then be rotated within keyway 442 to connect receiver 424 with connector 422. It is envisioned that distal portion 455 may be rotated between −135 and 135 degrees relative to keyway 442 to connect receiver 424 with connector 422.

Receiver 424 includes an insert, such as, for example, a second disk 450 having a first concave surface, such as, for example, an upper surface 472 that defines an opening 474 extending through second disk 450 configured for disposal of distal portion 455 of receiver 424. Upper surface 472 of second disk 450 has a channel 476 that has approximately the same diameter as an implant disposed in lateral opening 492 of receiver 424. The concavely curved bottom surface of lateral opening 492 is continuous with the concave shape of upper surface 472 of second disk 450 when receiver 424 is connected with second disk 450 as shown in FIG. 10 such that there are no gaps or protrusions between channel 476 and lateral opening 492. Second disk 450 includes one or more lateral openings 478 to accommodate a portion of the body portion of receiver 424. Thus, second disk 450 can slide onto receiver 424 so that second disk 450 fits relatively closely and/or snugly against receiver 424.

Receiver 424 includes a second surface 452 opposite upper surface 472 defined by axial splines 454 that mate with the splines on first splined surface 440 of first disk 436 to releasably fix receiver 424 with connector 422 in a selected rotatable position in a plane, such as, for example, a coronal plane of the body relative to shaft 28 of bone screw 20. Splines 454 and the splines on first splined surface 440 of first disk 436 are configured to mesh such that openings 490, 492 and second disk 450 can rotate and lock at different angles relative to connector 422 in the transverse plane such that a vertebral rod disposed in lateral opening 492 and posterior to first end 432 of connector 422 is selectively rotatable in a coronal plane of a body relative to first end 432 of connector 422 in a configuration for selective angular fixation with first end 432 of connector 422. It is envisioned that openings 490, 492 may be rotated through an angle of 0 to 360 degrees relative to connector 422. Second disk 450 is locked in position relative to first disk 436 by forcing disks 436, 450 into engagement.

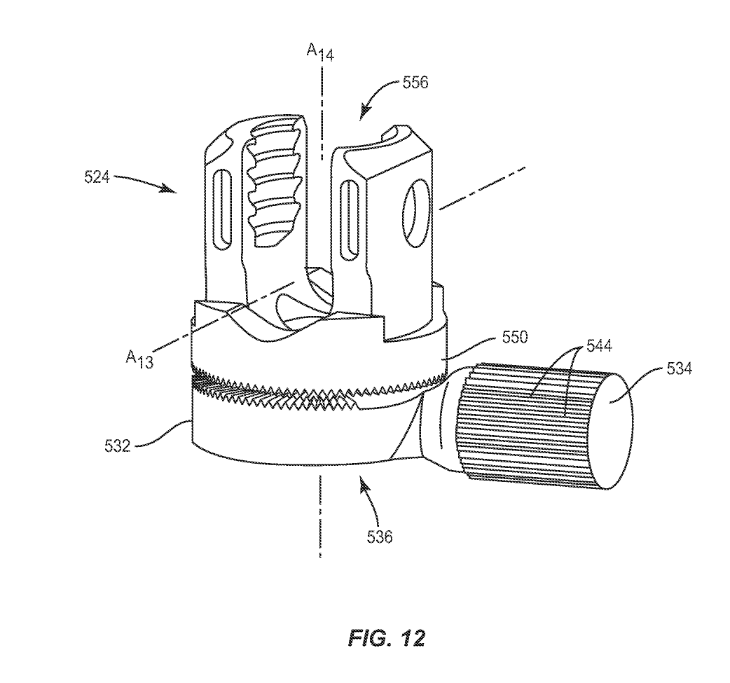

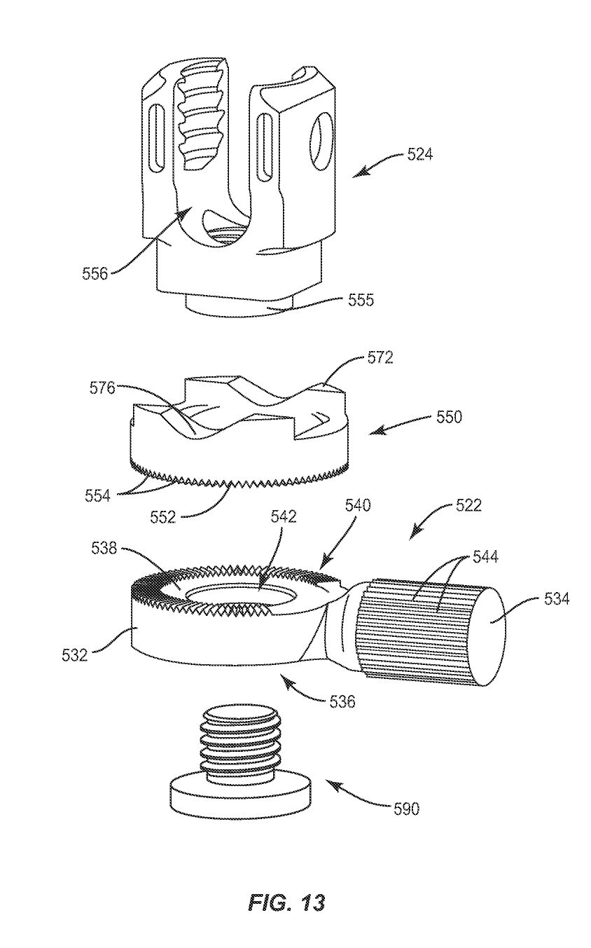

In one embodiment, as shown in FIGS. 12 and 13, the implant system includes bone screw 20 described above, a connector 522 and a receiver 524, similar to the components described above. Connector 522 extends between a first end 532 and a second end 534. First end 532 includes a first disk 536 having a first splined surface 540 configured to mate with splines on a surface of receiver 524 to releasably fix receiver 524 with connector 522 in a selected rotatable position in a plane, such as, for example, a coronal plane of the body relative to shaft 28 of bone screw 20. First end 532 of connector 522 includes a circumferential flange 538 defining a cavity 542 configured for engagement with a distal portion of receiver 524 such that the distal portion of receiver 524 is disposed within cavity 542 to retain receiver 524 with connector 522. First splined surface 540 has a height, which is greater than the height of circumferential flange 538 such that first splined surface 540 is superior to circumferential flange 538.

Second end 534 of connector 522 includes a cylindrical outer surface comprising axial splines 544 circumferentially disposed about the outer surface of second end 534. Axial splines 544 are engageable with axial splines 31 disposed about first cavity 30 of head 26 for selective angular fixation of the bone screw 20 with connector 522. Axial splines 544 include a plurality of individual spline members that extend in parallel relation about the outer surface of second end 534. The configuration of axial splines 544 provides at least a portion of a mounting and alignment configuration for aligning and mounting bone screw 20 with connector 522 and receiver 524 during a surgical treatment.

Second end 534 of connector 522 is configured for movement within first cavity 30 of bone screw 20 along axis A₁ in the coronal plane of the body for selective fixation in a position along axis A₁. Second end 534 of connector 522 is moveable along axis A₁ relative to shaft 28 of bone screw 20. It is envisioned that second end 534 of connector 522 may be inserted into first cavity 30 of bone screw 20. Second end 534 is moved within first cavity 30 in the coronal plane along axis A₁, in a first axial direction or a second axial direction oriented in an opposing direction to the first axial direction, by sliding second end 534 relative to first cavity 30. First cavity 30 is engageable with second end 534 to align the component parts in relative rotatable alignment.

Receiver 524 is attachable to first end 532 of connector 522 and is selectively rotatable in the transverse plane of the body and selectively fixable in a position within the transverse plane. Receiver 524 includes a body portion having an implant cavity 556 configured to receive and movably support at least a portion of an implant, such as, for example, a vertebral rod, such that the implant can translate axially within implant cavity 556. Implant cavity 556 defines an axis A₁₃ and is rotatable about an axis A₁₄ disposed transverse to axis A₁₃ such that an implant disposed in implant cavity 556 and posterior to first end 532 of connector 522 is rotatable in a plane, such as, for example, a coronal plane of the body, relative to first end 532 of connector 522 in a configuration for selective fixation with first end 532 of connector 522.

The body portion of receiver 524 includes a distal portion 555 configured to be received within cavity 542 of circumferential flange 538 such that distal portion 555 is disposed within cavity 542 to retain receiver 524 with connector 522. Distal portion 555 extends transversely from a bottom surface of receiver 524. Distal portion 555 is inserted into cavity 542 in connector 522 to connect receiver 524 with connector 522. Distal portion 555 includes a bore extending perpendicularly through a lower face of distal portion 555 configured to receive a retaining member, such as, for example, a setscrew to maintain the connection between receiver 524 and connector 522.

Receiver 524 includes a second disk 550 having a first concave surface, such as, for example, an upper surface 572 that defines an opening 574 (not shown) extending through second disk 550 configured for disposal of distal portion 555 of receiver 524. On upper surface 572 of second disk 550, a channel 576 is formed that is approximately the same diameter as an implant disposed in implant cavity 556. The concavely curved bottom surface of implant cavity 556 is continuous with the concave shape of channel 576 of second disk 550 when receiver 524 is connected with second disk 550 as shown in FIG. 12 such that there are no gaps or protrusions between channel 576 and implant cavity 556. Second disk 550 includes one or more lateral openings 578 to accommodate a portion of the body portion of receiver 524. Thus, second disk 550 can slide onto receiver 524 so that second disk 550 fits relatively closely and/or snugly against receiver 524.

Receiver 524 includes a second surface 552 opposite upper surface 572 defined by axial splines 554 that mate with the splines on first splined surface 540 of first disk 536 to releasably fix receiver 524 with connector 522 in a selected rotatable position in a plane, such as, for example, a coronal plane of the body relative to shaft 28 of bone screw 20. Splines 554 and the splines on first splined surface 540 of first disk 536 are configured to mesh such that receiver 524 (including implant cavity 556 and second disk 550) can rotate and lock at different angles relative to connector 522 in the transverse plane such that a vertebral rod disposed in implant cavity 556 and posterior to first end 532 of connector 522 is selectively rotatable in a coronal plane of a body relative to first end 532 of connector 522 in a configuration for selective angular fixation with first end 532 of connector 522. It is envisioned that implant cavity 556 may be rotated through an angle of 0 to 360 degrees relative to connector 522. Second disk 550 is locked in position relative to first disk 536 by forcing disks 536, 550 into engagement. Once receiver 524 is connected to connector 522 with second disk 550 positioned between receiver 524 and connector 522, a coupling member, such as, for example, a setscrew 590 is inserted through cavity 542 of connector 522 and opening 574 in second disk 550. Setscrew 590 is then inserted into the bore in the lower face of distal portion 555 of receiver 524 to maintain the connection between receiver 524, connector 522 and second disk 550.

In one embodiment, the implant system includes an agent, which may be disposed, packed or layered within, on or about the components and/or surfaces of the implant system. It is envisioned that the agent may include bone growth promoting material, such as, for example, bone graft to enhance fixation of the fixation elements with vertebrae.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of the implant system can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of the implant system. Upon completion of a procedure employing the implant system described above, the surgical instruments and assemblies are removed and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. An implant system comprising:
a fastener including a proximal portion and a distal portion, the proximal portion including an inner surface that defines a first cavity that defines a first axis, the distal portion being configured for penetrating tissue and defining a longitudinal axis disposed transverse to the first axis;
a connector extending between a first end and a second end configured for disposal in the first cavity; and
a receiver positioned posterior to the first end of the connector and attachable to the first end of the connector, the receiver including an implant cavity defining a second axis, the implant cavity being rotatable about a third axis disposed transverse to the second axis such that an implant disposed in the implant cavity and is positioned posterior to the first end of the connector and is rotatable in a first plane relative to the first end of the connector in a configuration for selective fixation with the first end of the connector.

2. The implant system of claim 1, wherein the first plane is a coronal plane of a body.

3. The implant system of claim 1, wherein the implant cavity is rotatable through an angle of 0 to 360 degrees.

4. The implant system of claim 1, wherein the implant is rotatable to a selected angular orientation for fixation with the first end of the connector.

5. The implant system of claim 1, wherein the inner surface of the proximal portion includes a splined configuration comprising axial splines oriented along the first axis and circumferentially disposed about the inner surface.

6. The implant system of claim 1, wherein the second end of the connector includes an outer surface including a splined configuration comprising axial splines circumferentially disposed about the outer surface of the second end of the connector.

7. The implant system of claim 1, wherein the inner surface of the proximal portion includes axial splines oriented along the first axis and circumferentially disposed thereabout and the second end of the connector includes axial splines circumferentially disposed thereabout, the axial splines of the proximal portion being engageable with the axial splines of the connector in a configuration for selective angular fixation of the fastener with the connector.

8. The implant system of claim 1, wherein the connector includes a first disk having a first splined surface, and the receiver includes a second disk having a second splined surface that mates with the first splined surface.

9. The implant system of claim 1, wherein the receiver includes a first locking part and the connector includes a second locking part, the first locking part being engageable for fixation with a surface of the second locking part to lock the receiver with the connector.

10. The implant system of claim 8, wherein the second locking part has a wishbone configuration and engages the first locking part to contact the surface of the second locking part.

11. The implant system of claim 1, wherein the receiver is tapered from an anterior end to a posterior end thereof.

12. The implant system of claim 1, wherein the receiver includes an anterior end having a first diameter that is greater than a second diameter of a posterior end thereof such that an inner surface of the second end of the connector is configured for close fitting engagement with the anterior end.

13. The implant system of claim 1, wherein the first end of the connector defines at least one keyway and the receiver defines at least one key configured for engagement therewith such that the at least one key is lockable with the at least one keyway to retain the receiver with the connector.

14. The implant system of claim 1, wherein the first end of the connector defines a pair of spaced apart arcuate keyways and the receiver includes a pair of spaced apart keys such that the keys are rotatable for locking engagement with the keyways to retain the receiver with the connector.

15. The implant system of claim 1, wherein the receiver includes an insert defining a concave first surface configured to receive the implant and a second surface comprising axial splines oriented along the third axis and circumferentially disposed about the second surface, the axial splines of the second surface being engageable with axial splines defined with an inner surface of the first end of the connector in a configuration for selective angular fixation of the implant with the connector.

16. The implant system of claim 1, wherein the receiver defines a posterior opening configured to receive the implant.

17. The implant system of claim 1, wherein the receiver defines a lateral opening configured to receive the implant.

18. The implant system of claim 1, wherein the receiver defines a threaded cavity oriented along the third axis and the connector includes a screw comprising a shaft disposable along the third axis, the screw being engaged with the threaded cavity to fix the receiver with the connector.

19. An implant system comprising:
an iliac bone screw including a posterior head and an anterior threaded shaft configured to penetrate bone, the posterior head including an inner surface that defines a first cavity that defines a first axis, the inner surface including axial splines oriented along the first axis and circumferentially disposed about the inner surface, the anterior threaded shaft defining a longitudinal axis disposed transverse to the first axis;
a connector extending between a first end and a second end, the first end including a first disk having a first splined surface, the second end including an outer surface comprising axial splines circumferentially disposed thereabout, the axial splines of the posterior head being engageable with the axial splines of the second end in a configuration for selective angular fixation of the iliac bone screw with the connector in a range of 30 to 150 degrees, the first end further including a first locking part comprising a circumferential flange; and
a receiver including a second disk having a first face defining an implant recess and a second face including a second splined surface that mates with the first splined surface, the receiver further including a second locking part including a first arm, a second arm and a wishbone part, the wishbone part being disposable to engage and force the arms into fixation with the circumferential flange to lock the receiver with the connector, the receiver further including an implant cavity defining a second axis,
wherein the implant cavity is rotatable about a third axis disposed transverse to the second axis such that a vertebral rod disposed in the implant cavity and posterior to the first end of the connector is selectively rotatable in a coronal plane of a body relative to the first end of the connector in a configuration for selective angular fixation with the first end of the connector.

20. A method for treating a disorder, comprising the steps of:
providing an implant system comprising:
a fastener including a proximal portion and a distal portion, the proximal portion including an inner surface that defines a first cavity that defines a first axis, the distal portion defining a longitudinal axis disposed transverse to the first axis,
a connector extending between a first end and a second end configured for disposal in the first cavity, and
a receiver positioned posterior to the first end of the connector and attachable to the first end of the connector, the receiver including an implant cavity defining a second axis, the implant cavity being rotatable about a third axis disposed transverse to the second axis;
attaching the distal portion of the fastener with iliac bone;
disposing an implant in the implant cavity such that the implant is posterior to the first end of the connector;
selectively rotating the implant in a first plane relative to the first end of the connector; and
locking the receiver with the connector in a selected angular orientation of the implant relative to the connector.
